(19) United States
(12) Patent Application Publication
(10) Pub. No.: US 2004/0126406 A1
(43) Pub. Date: Jul. 1, 2004

(54) Anatase-type titanium dioxide/organic polymer composite materials suitable for artificial bone

(76) Inventors: Tadashi Kokubo, Nagaokakyo-shi (JP); Shouichi Kawashita, Kyoto-shi (JP); Takeaki Miyamoto, Matsue-shi (JP)

Correspondence Address: SHERMAN & SHALLOWAY, 413 N. WASHINGTON STREET, ALEXANDRIA, VA, 22314, US

(21) Appl. No.: 10/475,256

(22) PCT Filed: Apr. 26, 2002
(86) PCT No.: PCT/JP02/04242

(30) Foreign Application Priority Data
May 2, 2001 (JP) 2001-135159
May 2, 2001 (JP) 2001-135161

(51) Int. Cl. A61K 33/24; A61F 2/00

(52) U.S. Cl. 424/423; 424/617

(57) ABSTRACT

Titanium dioxide/organic polymer hybrid materials for artificial bone produced by forming titania gel on the surface of a substrate made of an organic polymer and treating the titania gel with hot water or an aqueous solution of an acid to convert the titania gel into a titanium dioxide membrane on which apatite having such a Ca/P atomic ratio as to constitutes the bone of an mammal can be formed from the body fluid thereof. Specifically, a hybrid material composed of an organic polymer and crystallites of anatase type titanium dioxide which are bonded to each other on the molecular level, produced by condensing a titanium alkoxide through hydrolysis in the presence of a silanol-terminated organic polysiloxane and/or an alkoxysilyl-terminated polymer having a polyalkyrene oxide chain wherein the alkylene groups are represented by the formula: —(CH₂)n-, (wherein n is an integer of 1 or bigger) to form through a sol a hybrid composed of a polysiloxane or a polymer having a polyalkylene oxide chain wherein the alkylene groups are represented by the above formulaand titanium dioxide, and converting the titanium dioxide into crystallites of anatase-type titanium dioxide.

### FIELD OF THE INVENTION

The present invention basically relates to a titanium oxide/polymer hybrid material for an artificial bone. Concretely relates to a titanium dioxide-polymer hybrid material for an artificial bone characterizing on the surface of organic polymer substrate especially organic polymer containing hydroxyl group and/or derivatives thereof, thiol group, aldehyde group or amino group, a titanium dioxide layer having apatite forming ability, especially, having apatite forming ability in an aqueous solution with supersaturated inorganic ionic concentration or in a living body by lower temperature coating method of less than 300° C. is formed, or a bioactive organic/inorganic hybrid material with bioactivity, elastic modulus similar to a bone and high elongation obtained by bonding an organic polymer obtained by treating polymer-titanium oxide hybrid having polysiloxane obtained via sol prepared by hydrolysis/polycondensation of titanium alkoxide or polyalkyleneoxide chain possessing alkoxysilyl end and alkylene group represented by formula —(CH₂)n-, wherein n is an integer of 1 or bigger, so as to generate anatase type titanium oxide fine crystal with anatase type fine crystalline titanium dioxide by molecular level, under the presence of organic polysiloxane having silanol end and/or polymer having polyalkyleneoxide chain possessing alkoxysilyl end and alkylene group represented by formula —(CH₂)n-, wherein n is an integer of 1 or bigger.

### BACKGROUND OF THE INVENTION

The natural bone is a three dimensional hybrid composed of crystalline apatite linked by organic collagen fibers. Such kind of hybrid structure is formed by constructing three dimensionally collagen fibers of organic polymer on which inorganic apatite fine particles are regularly crystallized. Said organic collagen fibers acts mutual reinforcing function to apatite, and provides flexibility to a bone so as the bone to bend when outer pressure is loaded on it. If it is possible to form such mechanical structure three dimensionally using organic polymer fibers coated by apatite, the obtained hybrid becomes to have excellent bone bonding ability and mechanical properties similar to the natural bone. Therefore, it is useful as an apatite-polymer hybrid to compose hard tissue. And, the development of a novel material for artificial bone based on said view points is becoming popular.

Further, in various bones which compose a body, the required mechanical properties such as density, elastic modulus or elongation are different according to the part. Therefore, at the development of a practical bone substitute product, it is necessary to concern above mentioned factor.

In such a circumstances mentioned above,

A. The inventors of the present invention already reported that an uniform bonelike apatite layer with high density can be formed on an organic polymer, by an organic polymer is first set in contact with particles of a CaO—SiO₂-based glass in a simulated body fluid (SBF) with inorganic ion concentrations nearly equal to those of human plasma, then the organic polymer is dipped into another aqueous solution having 1.5 times inorganic ion concentrations to said SBF (hereinafter shortened to 1.5 SBF). In this case, in the first process, the silicate ion containing silanol (Si—OH) group released from said glass particles is attached to the polymer surface of polymer, then said Si—OH group forms apatite nuclei on the surface. At the second process, this apatite nuclei grows voluntarily absorbing calcium and phosphate ions from surrounding SBF. However, in this method, apatite is formed only at the polymer surface faced to glass particles.

B. Further, the inorganic material which is conventionally used as the bone substitute material, especially a composed material of silicon oxide with organic material, e.g. polysiloxane is used as the bone substitute material. As the material mentioned above, Wilkes reported a hybrid material obtained by the reaction of tetraethoxysilane with end silanol type polydimethylsiloxane (PDMS) (Polymer Preprints, pp300 vol.26, 1985). The inventers of the present invention also proposed a bone substitute material composed of a bioactive inorganic/organic hybrid material, in 1999 forum of Japan Ceramics Association held on Mar. 25-27, 1999 and in JP Patent Laid-Open Publication (Laid-opened on Mar. 27, 2001).

Along with said development for hybrid material useful as the bone substitute material with good plasticity, it become possible to provide the inorganic/organic hybrid material, the usability for the bone substitute material for a head bone or a jaw bone is improved.

The developments of an inorganic/organic hybrid material in said A and B are coincided at the point aiming to provide a hybrid material composed to have plasticity and mechanical strength of an organic polymer and apatite forming ability of an inorganic material organically so as the inorganic/organic hybrid material to have high bioactivity.

### DISCLOSURE OF THE INVENTION

The object of the present invention is basically relates to provide the technique which further improves the apatite forming ability in a body fluid of said A and B, and for the easy understanding the process for dissolving of the object, the present invention will be illustrated in relation with said prior arts A and B.

I Regarding the prior art A, the inventors of the present invention thought that if it is possible to introduce Si—OH group more uniformly on the surface of polymer, without using glass particles in solvent, it will be possible to provide a polymer material useful for the preparation of apatite-polymer hybrid with natural bone like three dimensional structure.

Based on above conception, as the first step, the inventors of the present invention carried out the investigation to find a polymer material which has affinity for forming a layer with high mechanical strength, easily forms apatite in SBF and shows high bioactivity. In said investigation, inventors of the present invention selected the organic polymer material containing ester group and/or hydroxyl group, in particular selected ethylene-vinyl alcohol copolymer (hereinafter shortened to EVOH). Further, the inventors of the present invention produced the substrate material prepared by denaturing the surface of said polymer by reacting with 3-isocyanatepropyl triethoxysilane [OCN(CH₂)₃Si(OC₂H₅)₃] (hereinafter shortened to IPTS) and silica solution. In the case of EVOH whose surface is denatured, it was possible to form apatite on the surface of it if 1.5 SBF (means simulated body fluid of 1.5 times inorganic ion concentrations) is used, however, in SBF of regular ion concentrations, apatite was not formed even after 21 day.

For the actual use of the substrate as an artificial bone, it is necessary that the bone like apatite is formed on the surface of the artificial bone, because the Ca/P atomic ratio of apatite formed in 1.5 SBF is far smaller than the Ca/P atomic ratio of apatite in natural bone.

Thereupon, the inventors of the present invention, carried out the investigation to further treat said IPTS treated EVOH with calcium silicate solution aiming to obtain more natural bone like artificial bone. The specimen treated with calcium silicate solution was confirmed that the apatite is formed in SBF even within 2 days. However, since calcium silicate gel layer formed on EVOH layer by said process is dissolved in SBF rapidly, it was difficult to form apatite with desired Ca/P atomic ratio on the surface of specimen whose surface is denatured.

In the investigation to provide the hybrid material composed of organic polymer and inorganic material useful as the artificial bone, the apatite with same Ca/P atomic ratio to that of natural born can be formed in supersaturated aqueous solution with respect to the apatite or in living body in controlled condition, the inventors of the present invention thought that the crystalline titania-organic polymer hybrid material whose surface is denatured by crystalline titania will be useful.

Recently, Uchida et al disclosed in the paper (M. Uchida, H. M. Kim, T. Kokubo and T. Nakamula, Bioceramics, 1999, vol.12, pp149-152) that Ti—OH group in titania gel having specific structure such as anatase causes the formation of apatite nuclei in short period in SBF. The solubility of titania gel in SBF is remarkably smaller compared with the solubility of calcium silicate gel to SBF.

Based on above mentioned conception, the inventors of the present invention thought that, when EVOH substrate whose surface is Si—OH denatured by above IPTS, and can control the structure of titania layer on EVOH substrate by followed hot water treatment, the obtained specimen becomes to have high apatite forming ability in supersaturated aqueous solution with respect to the apatite or in a living body.

The inventors of the present invention carried the trial to denature the surface of EVOH substrate by Ti—OH group using IPTS and titania solution. And the inventors of the present invention investigated the treatment with water or HCl aqueous solution of various concentrations aiming to control titania structure formed on above EVOH substrate by said surface denaturing treatment. In this trial, the inventors of the present invention found that on the surface of EVOH substrate treated by 0.10 M-HCl for five days large quantity of anatase is crystallized, and further found that on the surface of which anatase is crystallized apatite is formed in SBF within 14 days. Concerning this result, the inventors of the present invention thought that Ti—OH group in titania layer with anatase structure causes the formation of apatite nuclei on the surface of said EVOH substrate.

The inventors of the present invention investigated the apatite forming ability of the obtained specimen in SBF, changing the treating period variously from 0 day to 8 days for the purpose to make clear the relationship between the treating period by 0.10 M-HCl and apatite forming ability. Further, pH and/or treating temperature (temperature of hot water) in this treatment are also investigated. In these various investigations, the inventors of the present invention found that the concentration of acid, the treating period and the treating temperature (temperature of hot water) during the acid treatment after titania treatment are relating to the apatite forming ability of obtained substrate, and above mentioned object of the present invention based on the conception of A was dissolved.

By the way, the technique that the amorphous TiO₂—SiO₂ thin layer formed by sol-gel method can be converted to anatase structure by treating in hot water was proposed by Yoshinori Kotani et al (Journal of Sol-gel Science and Technology 19, 585-588, 2000), however, in this paper only photocatalyst function is referred but there is no refer about bioaffinity, especially, the use of it as the artificial bone is not referred at all.

II Regarding the prior art B, the object of the present invention is to provide a bone substituting material and a bone repairing material which are especially excellent in elastic modulus (has closer elastic modulus to a human cancellous bones), elongation to failure, tenacity and plasticity. Aiming to dissolve the object mentioned above, the inventors of the present invention found that the anatase type titanium oxide fine particles having apatite forming ability generates in a hybrid material which is prepared by following process, that is, an organic polymer possessing a reactive group at the end is coexisted at the hydrolysis/polycondensation process of a titanium oxide generating material so that forming sol solution, a hybrid material characterized by bonding titanium oxide and organic polymer in molecular level is prepared from said sol solution and by treating said hybrid material in hot water, thus the above mentioned object is dissolved.

### DISCLOSURE OF THE INVENTION

Therefore, the present invention based on the conception of afore mentioned A, is to provide a titanium oxide-organic polymer hybrid material for an artificial bone obtained by the process comprising, forming titania gel on the surface of a substrate substantially composed of an organic polymer then denaturing said taitania gel by treating with hot water or aqueous solution of acid to a titanium oxide membrane which forms apatite having same Ca/P atomic ratio to a bone of mammal from the body liquid of mammal. Desirably, the present invention is the titanium oxide-organic polymer hybrid material for an artificial bone, wherein the organic polymer contains hydroxyl group and/or derivatives thereof, thiol group, aldehyde group or amino group, more desirably the present invention is the titanium oxide-organic polymer hybrid material for an artificial bone, wherein the organic polymer composing the substrate is ethylene-polyvinyl alcohol copolymer. Further desirably, the present invention is the titanium oxide-organic polymer hybrid material for an artificial bone, wherein the substrate composed by the organic polymer is treated with a denaturing agent composed of a silane coupling agent which forms Si—OH group on the surface of said substrate, furthermore desirably, the present invention is the titanium oxide-organic polymer hybrid material for an artificial bone, wherein the silane coupling agent is the compound represented by general formula A.

R¹Si(—O—R²)(—O—R³)(—O—R⁴) general formula 1

(in general formula 1, R¹ is isocyanate group, epoxy group, vinyl group or hydro carbon group possessing chloride group, R², R³ or R⁴ are methoxy group or ethoxy group)

Still further desirably, the present invention is the titanium oxide-organic polymer hybrid material for an artificial bone, wherein the treating of said titania gel with hot water or aqueous solution of acid is carried out by the acid concentration of pH7 or less that forms titania membrane possessing Ti—OH group in anatase fine crystal and/or 1 hour to 1 month period and/or 30° C. to 120° C. temperature.

The present invention is anyone of the titanium oxide-organic polymer hybrid materials for an artificial bone mentioned above, wherein the apatite layer is foamed on the surface by contacting with supersaturated aqueous solution with respect to the apatite. The discovery that not only bioactivity is provided but also elastic modulus and elongation to failure are remarkably changed by said treatment with hot water is an excellent and not expected effect.

The first one of the present invention based on the conception of afore mentioned B, is a bioactive organic/inorganic hybrid material obtained by bonding an organic polymer obtained by treating polymer-titanium oxide hybrid having polysiloxane obtained via sol prepared by hydrolysis/polycondensation of titanium alkoxide or polyalkyleneoxide chain possessing alkoxysilyl end and alkylene group represented by formula —(CH₂)n- (n is an integer of 1 or bigger) so as to generate anatase type titanium oxide fine crystal with anatase type fine crystalline titanium dioxide by molecular level, under the presence of organic polysiloxane having silanol end and/or polymer having polyalkyleneoxide chain possessing alkoxysilyl end and alkylene group represented by formula —(CH₂)n- (n is an integer of 1 or bigger) or adding solvent in case of need. Desirably the present invention is the bioactive organic/inorganic hybrid material obtained by bonding said organic polymer with anatase type fine crystalline titanium dioxide by molecular level, wherein the treatment to generate anatase type titanium oxide fine crystal is to dip the substrate into hot water of 30° C. to 120° C. temperature or aqueous solution of acid, further the present invention is the bioactive organic/inorganic hybrid material wherein the apatite layer is formed on the surface by contacting with supersaturated aqueous solution with respect to the apatite. And the present invention is the use of these bioactive organic/inorganic hybrid material as a bone substitution material.

The second one of the present invention based on the conception of afore mentioned B, is the use of the bioactive organic/inorganic hybrid material obtained via sol prepared by hydrolysis/polycondensation of titanium alkoxide under the presence of organic polysiloxane having silanol end and/or polymer having polyalkyleneoxide chain possessing alkoxysilyl end and alkylene group represented by formula —(CH₂)n- (n is an integer of 1 or bigger) as a bone repairing material.

The third one of the present invention based on the conception of afore mentioned B, is a method for preparation of the bioactive organic/inorganic hybrid material comprising, bonding an organic polymer obtained by treating polymer-titanium oxide hybrid having polysiloxane obtained via sol prepared by hydrolysis/polycondensation of titanium alkoxide or polyalkyleneoxide chain possessing alkoxysilyl end and alkylene group represented by formula —(CH₂)n- (n is an integer of 1 or bigger) so as to generate anatase type titanium oxide fine crystal with anatase type fine crystalline titanium dioxide by molecular level, under the presence of organic polysiloxane having silanol end and/or polymer having polyalkyleneoxide chain possessing alkoxysilyl end and alkylene group represented by formula —(CH₂)n- (n is an integer of 1 or bigger) or adding solvent in case of need. Desirably, the present invention is the method for preparation of the bioactive organic/inorganic hybrid material characterized by bonding said organic polymer with anatase type fine crystalline titanium dioxide by molecular level, wherein the treatment to generate anatase type titanium oxide fine crystal is to dip the substrate into hot water or aqueous solution of acid.

The fourth one of the present invention based on the conception of afore mentioned B, is a method for preparation of the bioactive organic/inorganic hybrid material comprising, preparing sol or sol solution by hydrolysis/polycondensation of titanium alkoxide under the presence of organic polysiloxane having silanol end and/or polymer having polyalkyleneoxide chain possessing alkoxysilyl end and alkylene group represented by formula —(CH₂)n- (n is an integer of 1 or bigger) or adding solvent in case of need, generating polymer-titanium dioxide hybrid having polyalkyleneoxide chain possessing alkoxysilyl end and alkylene group represented by formula —(CH₂)n- (n is an integer of 1 or bigger), then preparing the bioactive organic/inorganic hybrid material characterized by bonding organic polymer with anatase type fine crystalline titanium dioxide by molecular level by the treatment to generate anatase type titanium oxide fine crystal and forming apatite on the surface of said bioactive organic/inorganic hybrid material by dipping into supersaturated aqueous solution with respect to the apatite.

### BRIEF ILLUSTRATION OF THE DRAWINGS

FIG. 1 shows XPS spectrum of untreated EVOH substrate (EVOH), IPTS treated EVOH substrate (IPTS-EVOH) and IPTS and titania treated EVOH substrate (IPTS-Ti-EVOH).

FIG. 2 shows TF-XRD observation patterns of the surface of EVOH substrate whose surface is treated by hot water by maximum 5 days after IPTS and titania treatment [0 day (1 d), 3 days (3 d), 5 days (5 d)], ● is EVOH and ♦ is anatase.

FIG. 3 shows the TF-XRD patterns of the surface of a specimen prepared by dipping the specimens of FIG. 2 into SBF by maximum 2 weeks [0 week (0 w), 1 week (1 w), 2 weeks (2 w)], wherein ● is EVOH and ♦ is anatase.

FIG. 4 shows XPS spectrum of the surface of EVOH substrate which is treated by IPTS and titania then treated by hot water for 5 days and further treated by 1.00M HCl (untreated=U-EVOH, treated=T-EVOH).

FIG. 5 shows the TF-XDR pattern of the substrate, whose surface is treated by IPTS and titania, then treated for 3 days (a) or 5 days (b) by hot water, further treated by 1.00M HCl, is dipped into SBF by maximum 2 weeks [0 week (0 w), 1 week (1 w), 2 weeks (2 w)], wherein ● is EVOH and ♦ is anatase.

FIG. 6 shows the XPS spectrum of EVOH substrate whose surface is treated by 0.00-1.00M HCl after IPTS and titania treatment [treat by 0M HCl (0.00M), treat by 0.01M HCl (0.010M), treat by 0.10M HCl (0.10M), treat by 1.00M HCl (1.00M)].

FIG. 7 shows the TF-XDR pattern of the substrate, whose surface is treated by IPTS and titania, then treated by 0.00-1.00M HCl [treat by 0M HCl (0.00M), treat by 0.01M HCl (0.010M), treat by 0.10M HCl (0.10M), treat by 1.00M HCl (1.00M)], wherein ● is EVOH and ♦ is anatase.

FIG. 8 shows the TF-XRD patterns of the surface of a specimen prepared by dipping the specimens of FIG. 7 [treat by 0M HCl (0.00M), treat by 0.01M HCl (0.010M), treat by 0.10M HCl (0.10M), treat by 1.00M HCl (1.00M)] into SBF for 2 weeks, wherein ● is EVOH, ♦ is anatase and ◯ is apatite.

FIG. 9 shows XPS spectrum (a) and TF-XRD pattern (b) of the surface of EVOH substrate which is treated for 1-8 days or not treated by 0.10M HCl [not treated (U), 1 day (1 d), 3 days (3 d), 5 days (5 d) , 8 days (8 d)] after treated by IPTS and titania, wherein ● is EVOH, ◯ is apatite and ♦ is anatase.

FIG. 10 shows the TF-XRD patterns of the surface of a specimen prepared by treating EVOH substrate, which is treated by IPTS and titania, with 0.10M HCl for 1 (b), 3 (c), 5 (d) and 8 days (e) and untreated substrate (a), then dipping in SBF for maximum 14 days [0 day (0 d) , 2 days (2 d), 4 days (4 d), 7 days (7 d) , 14 days (14 d)], wherein ● is EVOH, ♦ is anatase and ◯ is apatite.

FIG. 11 shows the thin film X ray diffraction pattern of the surface of PDMS-TiO₂ hybrid material (hereinafter shortened to PD10) treated by hot water for various periods [0 day (0 d), 1 days (1 d), 3 days (3 d), 7 days (7 d)] at 60° C. (a) and 80° C. (b), wherein ● is anatase and Δ is polymethylsiloxane.

FIG. 12 shows the thin film X ray diffraction pattern of the surface of specimen prepared by dipping PD10, which is treated by hot water for various periods [0 day (0 d), 1 days (1 d), 3 days (3 d), 7 days (7 d)] at 60° C. (a) and 80° C. (b), into SBF for 7 days, wherein ● is anatase and Δ is polymethylsiloxane.

FIG. 13 shows the stress (MPa)-strain (%) curve [before treatment (PT), after treatment (AT)] of PT10 hybrid material treated by hot water of 80° C. temperature for 7 days.

FIG. 14 shows the thin film X ray diffraction pattern of the surface of the specimen prepared by treating the hybrid material obtained by changing composing ratio (ratio by weight) of starting material of sol Si-PTMO/TiPT [PT30 (wt. ratio 30/70), PT40 (wt. ratio 40/60) and PT50 (wt. ratio 50/50)] by hot water, before treatment (PT), after treatment (AT) [2 days at 95° C. (95-2 d), 7 days at 80° C. (80-7 d)], wherein ● is anatase.

FIG. 15 shows the thin film X ray diffraction pattern of the surface of the specimen prepared by dipping PT30, PT40 and PT50 of FIG. 14 which are treated by hot water at 95° C. for 2 days (95-2 d) in SBF [before treatment (PT), treated for 1 day (1 d), 3 days (3 d), 7 days (7 d) , 14 days (14 d)], wherein ◯ is apatite and ● is anatase.

FIG. 16 shows the thin film X ray diffraction pattern of the surface of the specimen prepared by dipping PT30, PT40 and PT50 of FIG. 14 which are treated by hot water at 80° C. for 7 days (80-7 d) in SBF [before treatment (PT), treated for 1 day (1 d), 3 days (3 d), 7 days (7 d) , 14 days (14 d)], wherein ◯ is apatite and ● is anatase.

FIG. 17 shows the the stress (MPa)-strain (%) curve [before treatment (PT), after treatment (AT)] of PT40 of FIG. 14 which are treated by hot water at 95° C. for 2 days (95-2 d).

### THE BEST EMBODIMENT TO CARRY OUT THE INVENTION

The present invention will be illustrated more in detail. Regarding the present invention based on the conception of A.

I . The Preparation of a Substrate from the Starting Material for Substrate, Especially from EVOH.

As the material for forming a substrate, any organic polymer which has affinity to mammal such as human and can form a titania layer which has apatite forming ability in supersaturated aqueous solution with respect to the apatite can be used. As the material mentioned above, an organic polymer containing hydroxyl group and/or derivatives thereof, thiol group, aldehyde group or amino group can be mentioned as the desirable material, and can be used as the more desirable material by adjusting copolymerization ratio.

II. The Denaturation of a Substrate, Especially EVOH

EVOH substrate can be used as is, however, it is desirable to denature the surface of the substrate so as Si—OH group to be formed.

As the example of above mentioned material, a silane coupling agent represented by following general formula 1 (in general formula 1, R¹ is isocyanate group, epoxy group, vinyl group or hydro carbon group possessing chloride group, R², R³ or R⁴ are methoxy group or ethoxy group).

R¹Si(—O—R²)(—O—R³)(—O—R⁴) general formula 1

III. Even if the organic polymer material which can not form a titania layer having apatite forming ability in supersaturated aqueous solution with respect to the apatite as is such as polyolefin e.g. polyethylene or polypropylene can be a material for substrate composing a titanium oxide-organic polymer hybrid material of the present invention by using an organic group having affinity to polymer and a denature treating agent which forms Si—OH group.

In the present invention the wording of “substrate” indicates not only of a simple structure of plate or block but also indicates a concept containing complicated shape such as a bone of mammal.

IV. Example of the Treatment to Form Si—OH Layer on the Surface of Substrate, Especially a Treatment by IPTS.

In the nitrogen atmosphere, an EVOH substrate is dipped into silane solution composed of IPTS, dried toluene and di-n-butyltin diraurate. In particular, the EVOH substrate is dipped into silane solution of IPTS:toluene:di-n-butyltindiraurate=50:50:0.25 by weight ratio at 50° C. for 6 hours. After the reaction, the substrate is carefully washed by tetrahydrofurane, dried 2-propanol and dried toluene, then dried in vacuum condition for 24 hours. The obtained specimen is dipped into 0.05M-HCl of 40° C. temperature for 12 hours. The specimen picked out from said solution is dipped into D.I. water of 40° C. temperature for 12 hours, and further dried in vacuum condition at room temperature for 24 hours.

A silane coupling agent represented by general formula 1 such as vinyltrimethoxysilane or silanechrolide triisopropoxide can be used instead of IPTS.

V. Titania Treatment to Form a Titania Membrane

The mixture of super D.I. water, HNO₃ and C₂H₅OH anhydride is dropped slowly into the mixture of Ti(Oi-C₃H₇)₄ and C₂H₅OH anhydride at 5° C. and mixed so as to prepare titania solution of mole ratio of 1.0:0.1:0.1:9.25=Ti(Oi-C₃H₇)₄:H₂O:HNO₃:C₂H₅OH.

The obtained IPTS treated EVOH substrate is dipped into said taitania solution for 24 hours at the room temperature, picked up by 20 mm/minute rate, then dried at 100° C. for 10 minutes. This process is repeated for 4 times, then, finally the specimen is dried at 100° C. for 24 hours.

VI. Treatment by HCl Aqueous Solution to Provide Apatite Forming Ability to a Titania Layer.

(a) EVOH substrates treated by IPTS and titania are treated by 80° C. hot water for 5 days, and several specimens are treated by 1.00M HCl at 40° C. for 24 hours, then washed by super D.I. water at 40° C. for 24 hours (Example 1).

(b) EVOH substrates treated by IPTS and titania are treated by HCl aqueous solution by changing concentrations variously by maximum concentration of 1.00M at 80° C. for 5 days, then washed by super D.I. water at 40° C. for 24 hours (Example 2).

(c) EVOH substrates treated by IPTS and titania are treated by HCl aqueous solution by changing treating period variously by maximum 8 days at 80° C. for 5 days, then washed by super D.I. water at 40° C. for 24 hours (Example 3).

VII. Test of Apatite Forming Ability by Dipping into Simulated Body Fluid (SBF)

Obtained specimens are dipped into 30 ml of SBF adjusted to pH 7.40 and 36.5° C. temperature for various period by maximum 14 days. Specimen is picked up from said solution, washed carefully with super D.I. water, then dried at room temperature.

One example of supersaturated aqueous solution with respect to the apatite (simulated body fluid: SBF, with similar inorganic ion concentration to blood plasma of human)

[T. Kokubo, H. Kushitani, S. Sakka, T. Kitsugi and T. Yamamuro, “Solutions able to reduce in vivo surface-structure changes in bioactive glass-ceramic A-W”, J. Biomed, Master. Res.24, 721-734 (1966)]

In table 1, simulated body fluid (SBF) as the supersaturated with respect to the apatite and blood plasma of human are shown.

TABLE 1

Concentration/mM

| | Simulated | |
|---|---|---|
| ion | body fluid | Blood Plasma |
| Na⁺ | 142 | 142 |
| K⁺ | 5.0 | 5.0 |
| Mg²^{⁺} | 1.5 | 1.5 |
| Ca²⁺ | 2.5 | 2.5 |
| Cl⁻ | 148 | 103.0 |
| HCO₃⁻ | 4.2 | 27.0 |
| HPO₄²⁻ | 1.0 | 1.0 |
| SO₄²⁻ | 0.5 | 0.5 |

Further, regarding the present invention based on the conception of B. At the preparation of sol or sol solution by hydrolysis/polycondensation of titanium alkoxide, it is important to carry out the preparation under the presence of an organic polymer having a group reactive to a generating titania sol at least at two ends, has affinity to body fluid and does not have rejection.

As the example of mentioned organic polymer (including oligomer), organic polysiloxane with silanol end or polymer with mono, di or tri alkoxysilyl end and polytetraalkyleneoxide chain can be mentioned as the desirable example. By introducing a reactive group at both ends of polyolefin, an olefin chain can be introduced.

VIII . As Titaniumalkoxide, Tetraethyltitanete (TEOT) or Tetraisopropyltitanate (TiPT) can be Mentioned as the Desirable Example.

Further, at the preparation of sol or sol solution, when fibers having high elastic modulus such as organic polymer, glass, carbon or silicon carbide are added, the elastic modulus and mechanical intensity of hybrid material can be improved.

IX. Test of Apatite Forming Ability of the Obtained Specimen and Analysis of the Surface Structure.

1. Test of apatite forming ability by dipping into simulated body fluid (SBF); Specimen is dipped into 30 ml of SBF adjusted to pH 7.40 and 36.5° C. temperature for various period by maximum 14 days, then said specimen is picked up from said solution, washed by super D.I. water carefully, dried at room temperature,

2. measured by X ray photoelectron spectroscopy (XPS:MT-5500, product of ULVAC-PHI Co., Ltd.) and by

3. thin film X ray diffracting meter (TF-XRD:RINT2500, product of Rigaku).

X. Measurement of Mechanical Properties of the Obtained Specimen:

Measured by Autograph (bending strength : AGS-10KNG, product of Shimazu Seisakusho, and elongation: product of Shimazu Seisakusho)

### EXAMPLE

The present invention will be illustrated more in detail along with the Examples. However, following Examples are mentioned for the purpose to make clear the usefulness of the present invention, and not intending to limit the scope of the claim of the present invention.

### Example 1

1. Preparation of EVOH substrate; EVOH (product of Kuraray Co., Ltd.) of ethylene contents is 32 mol % is molded by a hot press and a plate shape specimen of 1 mm thickness and 10 mm square is cut off from it and ground by #400 diamond abrasive plate. The specimen is rinsed by acetone and 2-propanol, then dried in vacuum condition at 100° C. for 24 hours, thus the EVOH substrate is prepared.

2. The surface of said substrate is treated by 3-isocyanate propyltriethoxy silane (ITS), then by treating with HCl, Si—OC₂H₅ group is hydrolyzed and converted to Si—OH. After HCl treatment, the specimen is dried by vacuum at the room temperature (hereinafter, shortened to IPTS treated EVOH).

3. The mixed solution of super D.I. water, HNO₃, C₂H₅OH anhydride is dropped slowly into the mixture of Ti(Oi-C₃H₇)₄ and C₂H₅OH anhydride at 0° C. and mixed so as to prepare titania solution of mole ratio of 1.0:1.0:0.1:9.25=Ti(Oi-C₃H₇)₄:H₂O:HNO₃:C₂H₅OH. Said EVOH substrate treated by IPTS is dipped into said taitania solution for 24 hours at the room temperature, then picked up by 20 mm/minute rate and dried at 100° C. for 10 minutes. This process is repeated for 4 times, then, finally the specimen is dried at 100° C. for 24 hours.

4. After that, the specimen is treated by dipping into hot water of 80° C. for various period by maximum 5 days. Several specimens are further treated by 1.00M HCl at 40° C. for 24 hours, and treated by super D.I. water at 40° C. for 24 hours.

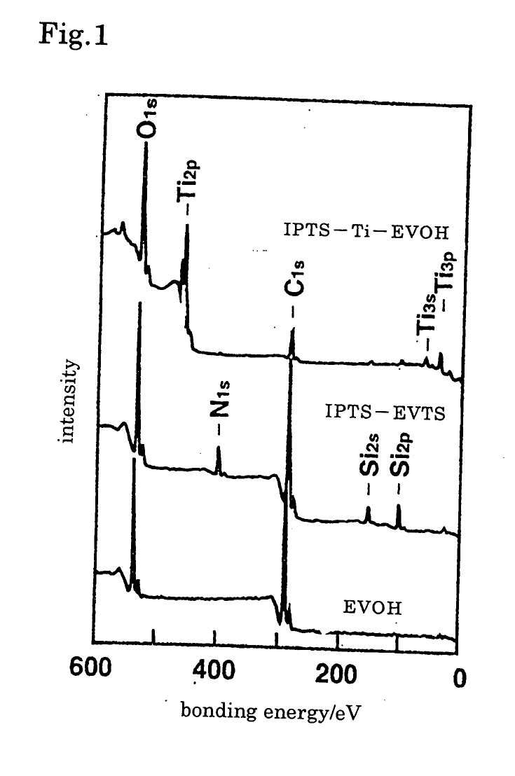

In FIG. 1, XPS spectrum of untreated EVOH substrate (EVOH), IPTS treated EVOH substrate (IPTS-EVOH) and IPTS and titania treated EVOH substrate (IPTS-Ti-EVOH) are shown. In EVOH substrate treated by IPTS, peaks based on N₁ₛ, Si₂ₛ and Si₂ₚ are observed. This observation indicates that IPES is existing on a specimen. After titanum treatment, new peaks based on Ti₂ₚ, Ti₃ₛ and Tiₛₚ are observed. This fact indicates that a titania layer is formed on IPTS treated EVOH substrate.

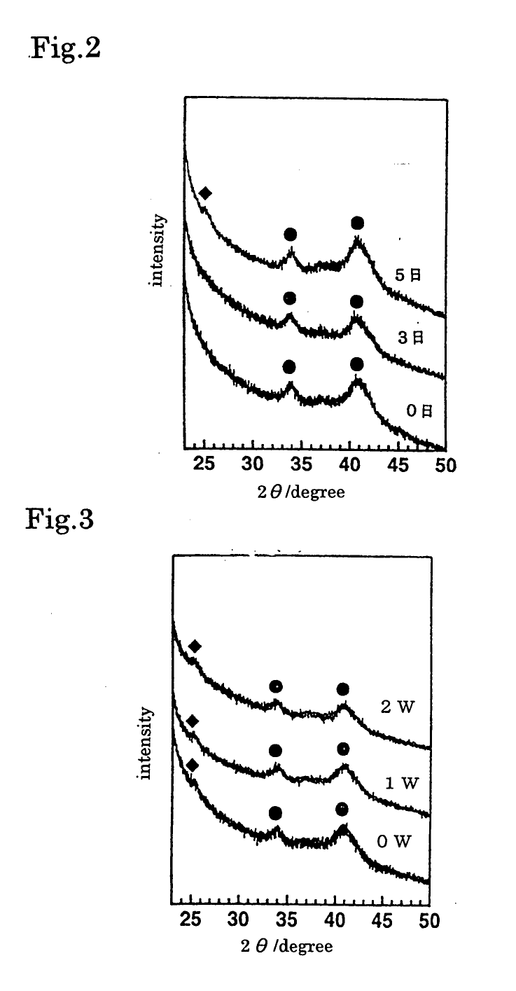

TF-XRD observation patterns of the surface of EVOH substrate whose surface is treated by hot water for various periods [0 day (1 d), 3 days (3 d), 5 days (5 d)] by maximum 5 days after IPTS and titania treatment are shown in FIG. 2. From the specimens of the hot water treatment period of 0-3 days [0 day (1 d), 3 days (3 d), 5 days (5 d)] two peaks originated to EVOH, that is, peaks at approximately 34° and 41° are observed. On the contrary, from the specimen treated by hot water for 5 days, small peak originated to anatase structure is observed. This observation result indicates that the amorphous titania gel layer formed on EVOH changed to anatase structure by hot water treatment for 5 days.

5. The apatite forming ability of the specimen obtained by the condition mentioned in VII (maximum 2 weeks) is investigated by dipping it into SBF shown in Table 1. Whether apatite is formed or not is investigated using an apparatus mentioned in IX.

As clearly understood from FIG. 3, the specimen which is treated by hot water for 5 days does not form apatite on the surface even if dipped into SBF for 2 weeks (2 w). The reason of this result is thought that the amount of Ti—OH formed on the surface is small and the surface of gel indicates hydrophobicity effected by alkoxyl group remained in the surface layer.

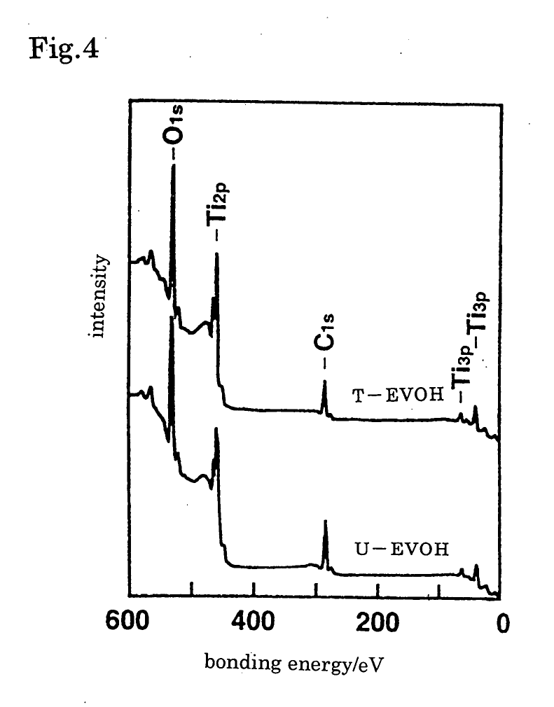

6. Aiming to change alkoxy group remained on the surface of specimen to Ti—OH group by hydrolysis, the specimen is further treated by 1.00M HCl. XPS spectrum of the surface of EVOH substrate which is treated by IPTS and titania then hot water for 5 days and further treated by 1.00M HCl (T-EVOH) is shown in FIG. 4, while, not treated specimen by 1.00M HCl is shown by (U-EVOH). The relative peak intensity of carbon to titanium of the treated specimen by HCl becomes smaller compared with that of untreated specimen. This result indicates that the numbers of Ti—OH group on the specimen increased by HCl treatment.

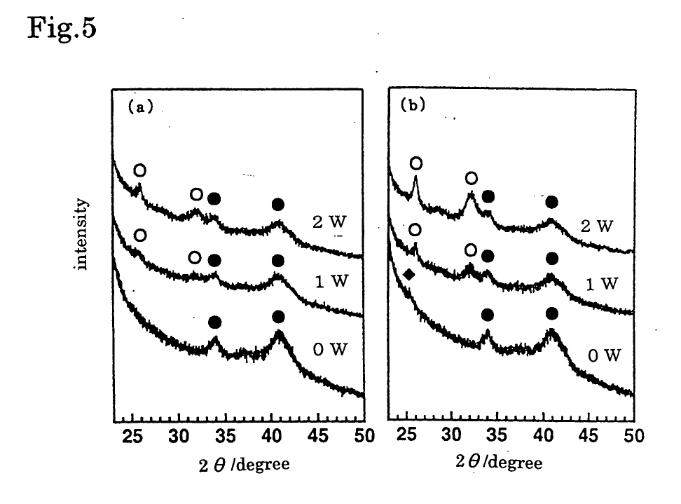

7. TF-XDR patterns of the substrate, whose surface is treated by IPTS and titania, then treated by hot water for 3 days [FIG. 5 (a)] or 5 days [FIG. 5 (b)], further treated by 1.00M HCl, and is dipped into SBF by maximum 2 weeks [0 week (0 w), 1 week (1 w), 2 weeks (2 w)] are shown in FIG. 5. By the results, it becomes clear that the substrate treated by IPTS and titania, then treated by hot water for 3 days (a) or 5 days (b), further treated by 1.00M HCl forms apatite (◯) on the surface in SBF within one week. By many Ti—OH groups formed on the surface of specimen, apatite is formed in SBF. The peak intensity of apatite of the specimen treated for 5 days is stronger compared with that of the specimen treated for 3 days. The reason of this result is thought that the crystallized amount of apatite in former is larger than that of latter, because Ti—OH group on titania gel with anatase structure has high apatite forming ability compared with Ti—OH group on amorphous titania gel.

### Example 2

1. Preparation of EVOH Substrate

EVOH (product of Kuraray Co., Ltd.) of ethylene contents is 32 mol % is molded by a hot press and a plate shape specimen of 1 mm thickness and 10 mm square is cut off from it and ground by #400 diamond abrasive plate. The specimen is rinsed by acetone and 2-propanol, then dried in vacuum condition at 100° C. for 24 hours, thus the EVOH substrate is prepared.

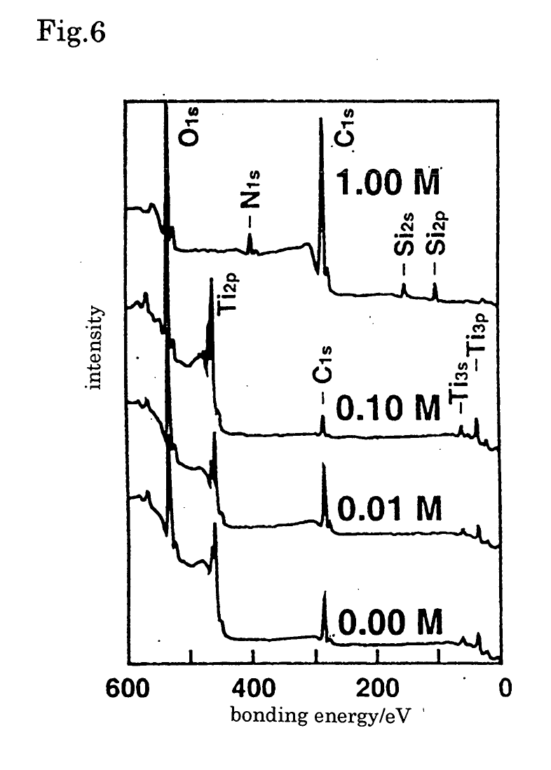

XPS spectrum of EVOH substrate whose surface is treated by 0.00-1.00M HCl after IPTS and titania treatment [treat by 0M HCl (0.00M), treat by 0.01M HCl (0.010M), treat by 0.10M HCl (0.100M), treat by 1.0M HCl (1.00M)] is shown in FIG. 6. In the case of EVOH substrate whose surface is treated by HCl of lower concentration than 1.00M after IPTS and titania treatment, a peak according to Ti is observed. While, in the case of a specimen treated by 1.00M HCl, a peak according to Ti is not observed. The spectrum by this specimen is very similar to that of spectrum of IPTS treated EVOH substrate in FIG. 1 (IPTS-EVOH).

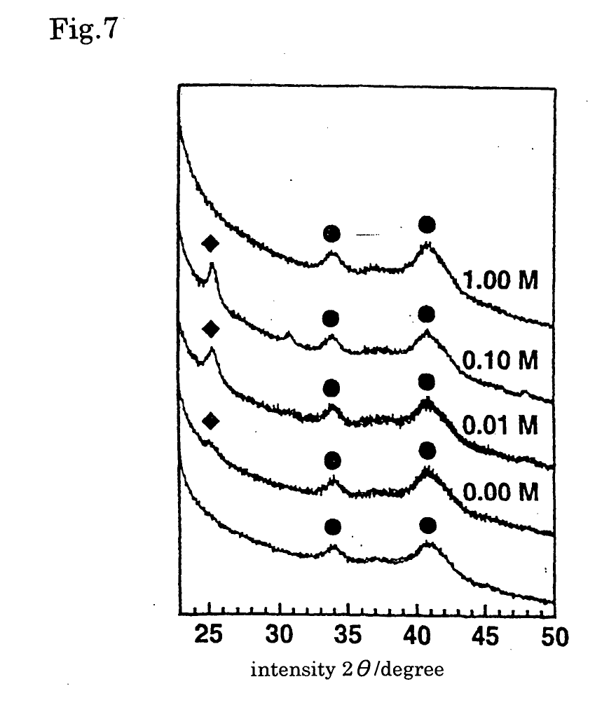

In FIG. 7 the TF-XDR pattern of the substrate, whose surface is previously treated by IPTS and titania, then treated by 0.00-1.00M HCl is shown. In the case of IPTS and titania treated EVOH (0.00M), only two peaks at approximately 34° and 41° are observed. From this result, the titania layer formed on the specimen is understood as amorphous. In the case of specimens treated by HCl smaller than 1.00 concentration [treated by 0.01M HCl (0.010M), treated by 0.10M HCl (0.10M), treated by 1.00M HCl (1.00M)], a peak originated to anatase (♦) is observed. The intensity of peak originated to anatase increases along with the increase of HCl concentration till the concentration of HCl becomes 0.10M. These results indicate that the amorphous titania gel layer changes to anatase structure by the treatment by HCl aqueous solution of 0.00-0.10M concentration. In the case of the specimen treated by 1.00M-HCl, the peak originated to anatase is not observed. The reason why the peak originated to anatase is not observed, is illustrated from the XPS result of FIG. 6, that is, because the titania layer formed on the specimen is dissolved by 1.00M HCl.

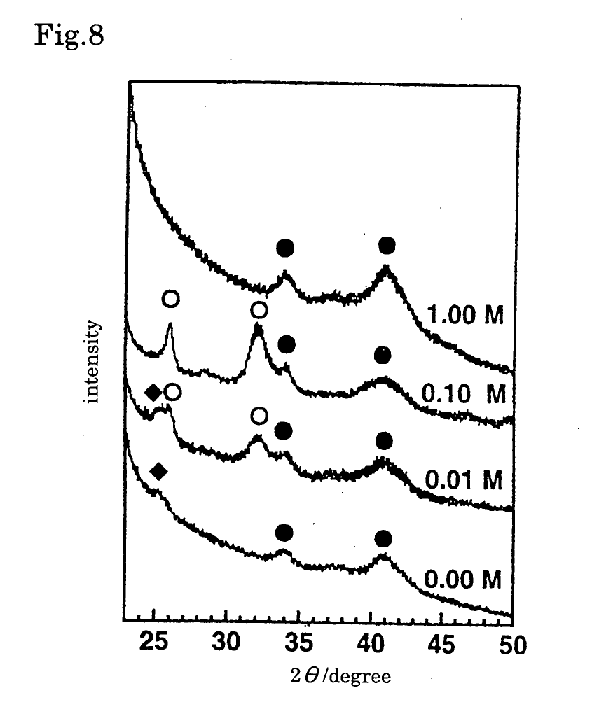

In FIG. 8 the TF-XRD patterns of the surface of specimen prepared by dipping the specimens of FIG. 7 whose surface is previously treated by IPTS and titania, then treated by 0.00-1.00M HCl into SBF for 2 weeks are shown. In the cases of the specimen treated by 0.01 and 0.10 HCl, the peak originated to apatite (◯) is observed. Regarding the peak originated to apatite, the height of the peak of the specimen treated by 0.10M HCl is higher than that of the specimen treated by 0.01M HCl. These results indicates that 0.01-0.10M HCl treated substrate which is previously treated by IPTS and titania forms apatite on the surface in SBF within 2 weeks, and the apatite forming ability of the specimen is improved along with the increase of HCl concentration till 0.10M HCl concentration. The reason of this result is thought that the crystallized amount of anatase by HCl treatment on specimen increases along with the increase of HCl concentration untill the concentration of HCl increases to 0.10M.

From these results, the specimen which has good apatite forming ability can be obtained when 0.01M HCl aqueous solution is used.

### Example 3

1. Preparation of EVOH Substrate;

EVOH (product of Kuraray Co., Ltd.) of ethylene contents is 32 mol % is molded by a hot press and a plate shape specimen of 1 mm thickness and 10 mm square is cut off from it and ground by #400 diamond abrasive plate. The specimen is rinsed by acetone and 2-propanol, then dried in vacuum condition at 100° C. for 24 hours, thus the EVOH substrate is prepared.

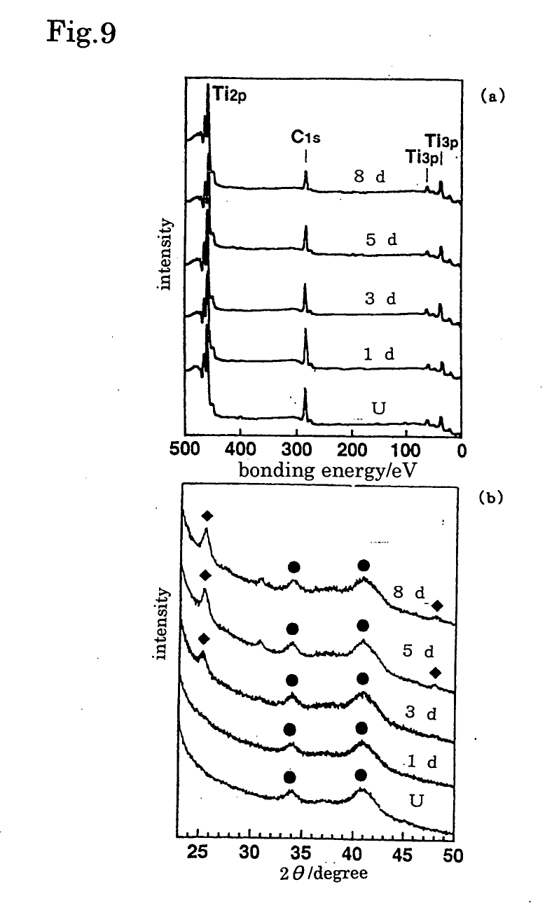

In FIG. 9 shows XPS spectrum [FIG. 9(a)] of the surface of EVOH substrate which is treated for 1-8 days and not treated by 0.10M HCl [not treated (U), 1 day (1 d), 3 days (3 d), 5 days (5 d) , 8 days (8 d)] after treated by IPTS and titania solution. In XPS spectrums, peaks based on C₁ₛ, Ti₂ₚ, Ti₃ₛ and Tiₛₚ are observed in all specimens. This result shows that a titania layer is existing on the surface of specimens. The relative peak intensity of C₁ₛ against Ti₂ₚ decreases along with the increase of the treating period by HCl. This phenomenon can be explained as follows. That is, in a titania layer formed on the surface of the substrate which is previously treated by IPTS and titania many alkoxy groups are contained. This is because the drying temperature at the titania treatment is low (100° C.). When said specimen is treated by HCl, alkoxy group existing on the surface of specimen is hydrolyzed by catalytic effect of HCl and changed to Ti—OH group.

In the case of specimen which is not treated by HCl or treated by HCl for one day, only two peaks at approximately 34° and 41° based on EVOH in TF-XRD pattern [FIG. 9(b)] are observed. From these results, the structure of titania formed on these specimens is understood to be mainly amorphose. In the specimen treated by HCl for 3-8 days, a peak based on anatase is observed.

The peak intensity by anatase becomes stronger along with the increase of the treating period by HCl. These results indicate that the amorphous titania layer changes to anatase by the treatment by HCl and the crystallized amount of anatase increases along with the increase of the treating period by HCl until maximum 8 days.

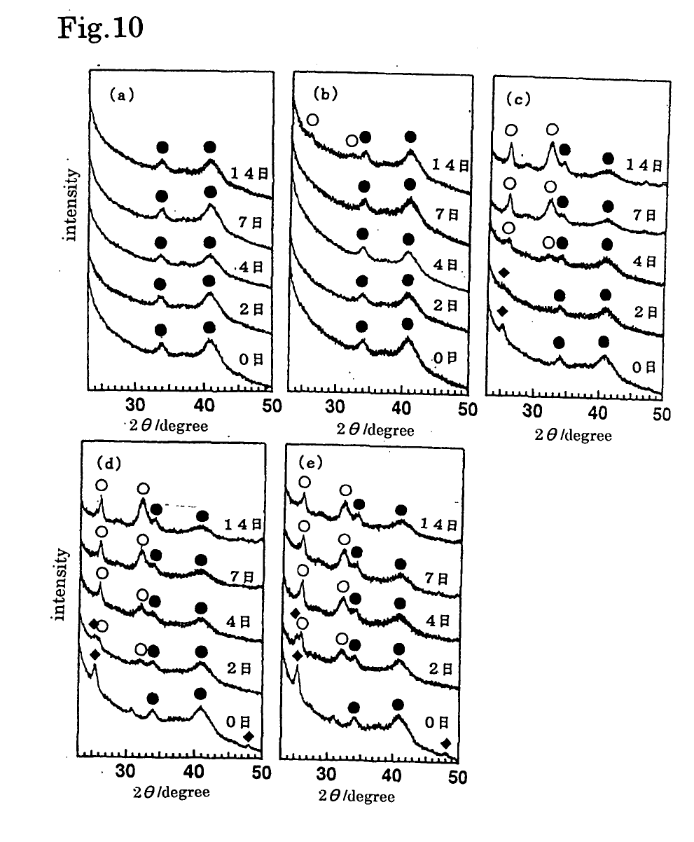

In FIG. 10 the TF-XRD patterns of the surface of specimen prepared by treating EVOH substrate, which is previously treated by IPTS and titania, with 0.10M HCl for 1 (b)-8 days (e) and untreated substrate (a), then dipped into SBF for maximum 14 days [0 day (0 d) , 2 days (2 d), 4 days (4 d), 7 days (7 d), 14 days (14 d)] are shown. In the case of untreated specimen (a), a peak based on apatite is not observed. While, in the case of specimens treated by HCl for 1 (b), 3 (c), 5 (d) and 8 (e), a peak based on apatite is observed relatively after 14, 7, 4 and 2 days from the dipping day in SBF. The peak intensity by anatase of 8 days HCl treatment is stronger than that of 5 days HCl treatment. These results indicates that HCl treated substrate for 1-8 days which is previously treated by IPTS and titania forms apatite on the surface in SBF within 2 weeks, and the period required for the formation of apatite can be shortened to two days along with the increase of the treating period by HCl. The reason why can be considered that the Ti—OH group in titania layer of anatase structure causes the formation of apatite in SBF.

The material on the surface of which an apatite layer is formed by the contact with supersaturated aqueous solution with respect to the apatite is useful as the material for an artificial bone.

Above mentioned Examples are relating the invention based on the conception of A.

### Example 4

Tetraethyltitanate (TEOT), ethyl acetoacetate (EAcAc) and ethanol (EtOH) are mixed and stirred, and polymethylsiloxane (PDMS) of 550 molecular weight is added and mixed with stirring. Further, water (H₂O) and ethanol are added and stirred, and sol solution is prepared. Each components are blended so as the component of sol to be PDMS/TEOT (Si/Ti)=1.36, EAcAc/TEOT=2, H₂O/TEOT=2 and EtOH/TEOT=8.

The obtained sol solution is contained into a container made of tetrafluoroethylene and covered by an aluminium foil with small holes and left for 2 days at 70° C. so that allow the gelation. Thus the precursor for PDMS-TiO₂ hybrid material is obtained. This precursor is treated by heat at 100° C. for 2 days, further at 150° C. for 3 days and PDMS-TiO₂ hybrid material (shortened to PD10) is obtained.

The obtained PDMS-TiO₂ hybrid material is contained into a container with D.I. water of 80° C. and dipped, then treated by hot water by shaking the container, and anatase type titanium oxide crystalline fine particles-PDMS hybrid material (shortened to hot water treated PD10) is obtained.

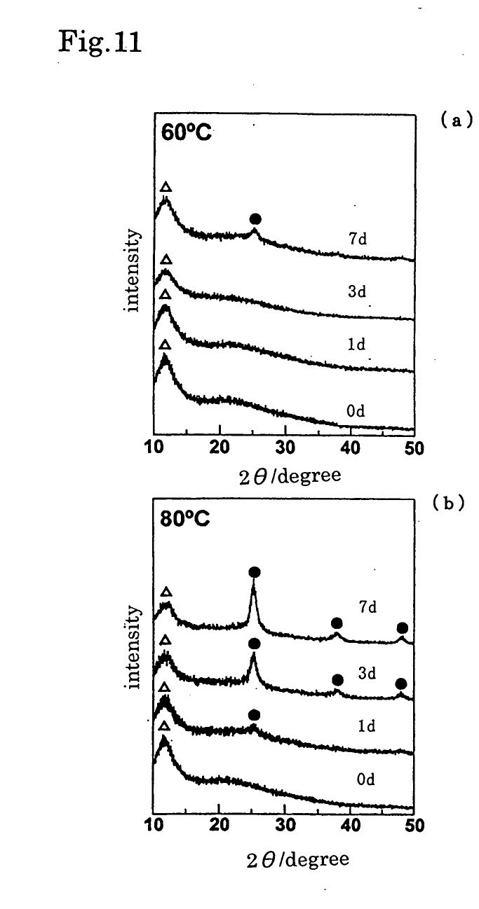

FIG. 11 shows the. thin film X ray diffraction pattern of the surface of PD10 treated by hot water of 60° C. (a) or 80° C. (b). These specimen are dipped in the simulated body fluid (pH7.40, temperature 36.5° C.).

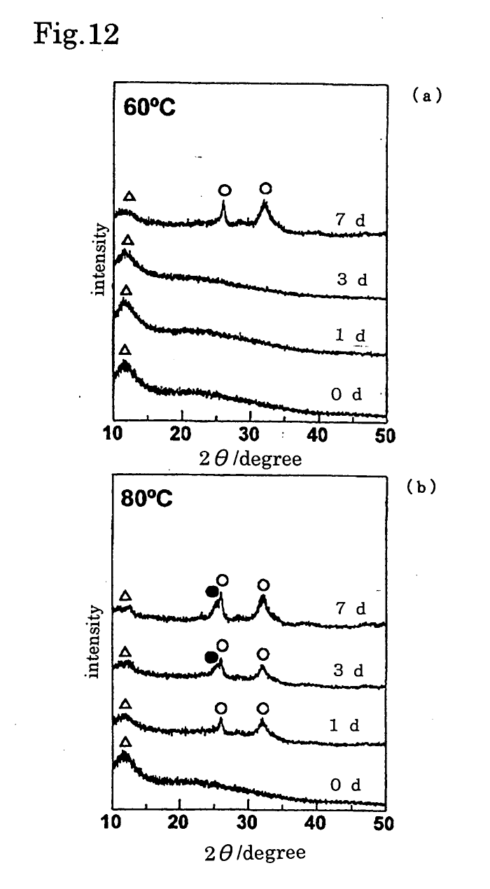

The thin film X ray diffraction pattern of the surface of specimen prepared by dipping PD10, which is treated by hot water for various periods [0 day (0 d), 1 days (1 d), 3 days (3 d), 7 days (7 d)] into simulated body fluid for 7 days, is shown in FIG. 12 [temperature 60° C. (a), temperature 80° C. (b)]. The generation of apatite (◯) is confirmed.

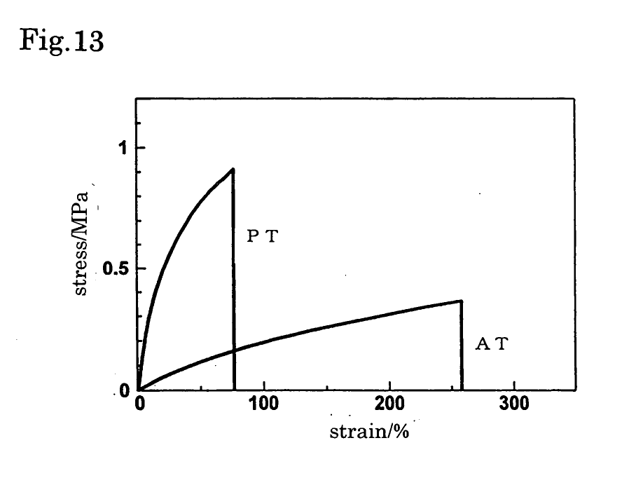

Test piece for tensile test (AT) (2 mm width×1-2 mm thickness×15 mm length) is cut out from PDMS-TiO₂ hybrid material which is treated by hot water of 80° C. temperature for 7 days. Said test piece is tested by a tension testing machine by 2 mm/minute stretching speed. Stress (MPa)-strain (%) curve is measured [test piece cut out from the specimen before treatment (PT) is used as the specimen for comparison] (FIG. 13).

From the result, the improvement of elongation for failure by hot water treatment is confirmed.

### Example 5

Tetraethyltitanate (TEOT), ethyl acetoacetate (EAcAc) and ethanol (EtOH) are mixed and stirred, and polymethylsiloxane (PDMS) of 550 molecular weight is added and mixed with stirring. Further, water (H₂O) and ethanol are added and stirred, and sol solution is prepared. Components ratio and name of specimens are shown in Table 2.

Si-PTMO can be obtained by reacting polytetramethyleneoxide (PTMO) [HO—(CH₂)₄—O)n-H] with 2 mole of 3-isocyanatepropylethoxysilane [(C₂H₅O)₃Si(CH₂)₃NCO].

TABLE 2

| | component | | |
|---|---|---|---|
| | Si-PTMO/TiPT | H₂O/Tipt | HCI/TiPT |
| name | (weight ratio) | (molar ratio) | (molar ratio) |
| PT30 | 30/70 | 2 | 0.05 |
| PT40 | 40/60 | 2 | 0.05 |
| PT50 | 50/50 | 2 | 0.05 |

Said sol solution is contained in a container made of tetrafluoroethylene and covered by an aluminium foil with small holes and the precursor of PTMO-TiO₂ hybrid material is obtained by gelation for 4 weeks and by drying.

The obtained precursor of PTMO-TiO₂ hybrid material is contained into a container with D.I. water of 80° C. or 95° C. and dipped, in the former case for 7 days and in latter case for 2 days, and completed the treatment by hot water. Thus the PTMO-TiO₂ hybrid material of the present invention is obtained [at 95° C., for 2 days (95-2 d), at 80° C., for 7 days (80-7 d)].

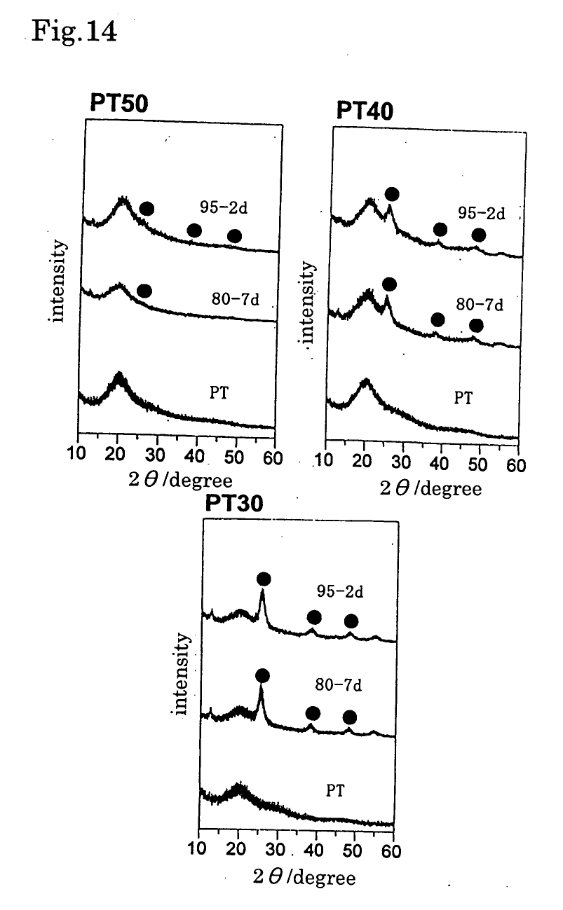

The thin film X ray diffraction patterns of the surface of PT30, PT40 and PT50 before treatment by hot water and PTMO-TiO₂ hybrid material after treatment by hot water are shown in FIG. 14. From the result, the generation of anatase type TiO₂ (●) is confirmed.

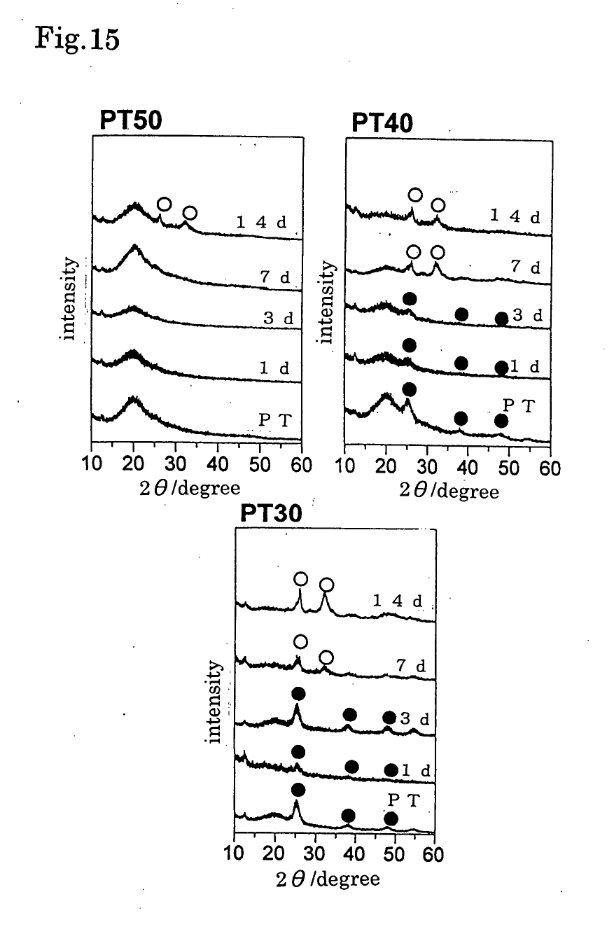

The thin film X ray diffraction patterns of the surface of the specimen prepared by dipping PTMO-TiO₂ hybrid material which is treated by hot water at 95° C. for 2 days (95-2 d) into simulated body fluid mentioned in Table 1 during several periods [before treatment (PT), treated for 1 day (1 d), 3 days (3 d), 7 days (7 d), 14 days (14 d)] are shown in FIG. 15. The generation of apatite (◯) is confirmed.

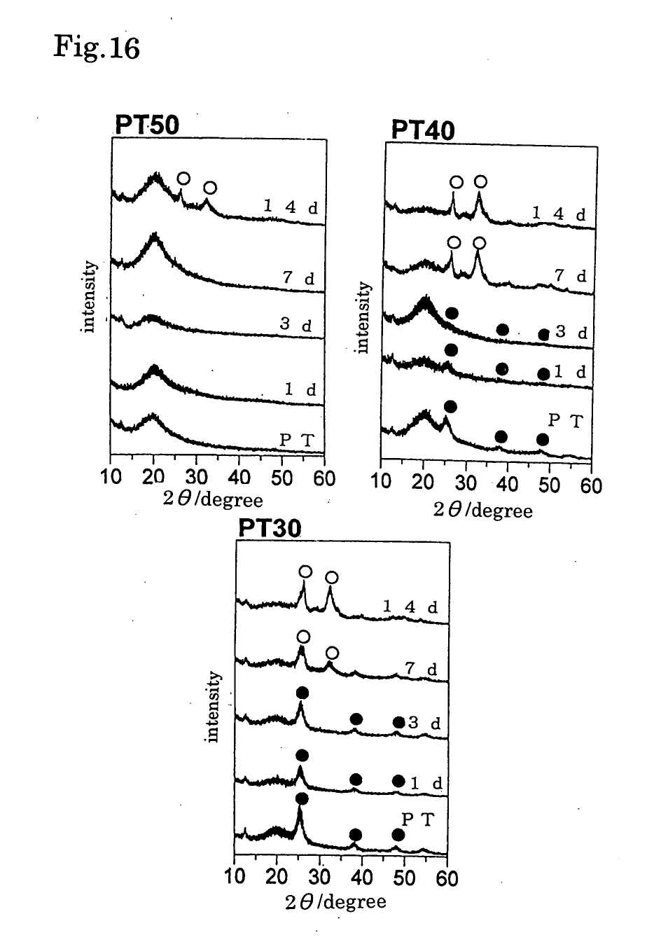

The thin film X ray diffraction pattern of the surface of the specimen prepared by dipping PTMO-TiO₂ hybrid material which is treated by hot water at 80° C. for 7 days (80-7 d) into simulated body fluid mentioned in Table 1 during several periods [before treatment (PT), treated for 1 day (1 d), 3 days (3 d), 7 days (7 d), 14 days (14 d)] are shown in FIG. 16. The generation of apatite (◯) is confirmed.

Test piece of 3×4×30 mm² [before treatment by hot water (PT), after treated (AT)] is prepared from the obtained specimen and said test piece is tested by a bending machine by the condition of width 3 mm×thickness 4 mm×distance 15 mm and crosshead speed 0.5 mm and stress(MPa)-strain (%) feature is measured.

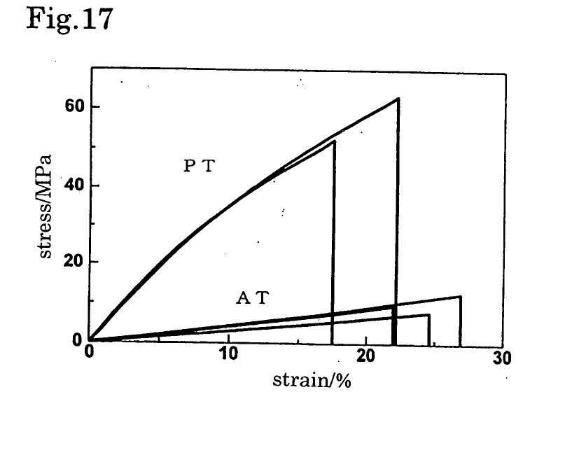

The results are shown in FIG. 17. Bending strain feature is improved. Above mentioned Examples 4 and 5 are relating the invention based on the conception of B.

Possibility for the Industrial Use

As mentioned above, the titania layer formed based on the conception of A has excellent effects from the point that the apatite forming ability is improved to the level for the actual use as the artificial bone and from the point that the present invention provides a titania-organic polymer hybrid suited to substantial artificial bone. Further, anatase type titanium dioxide-organic polymer hybrid material formed based on the conception of B has an excellent effect that said material provides an organically hybrid material whose mechanical intensity feature and bioactivity are improved simultaneously, namely, the bioactivity as a bone substitution material and a bone repairing material and the elongation to failure (high elongation) are improved.

What is claim

1. A titanium oxide-organic polymer hybrid material for an artificial bone obtained by the process comprising, forming titania gel on the surface of a substrate substantially composed of an organic polymer then denaturing said titania gel by treating with hot water or aqueous solution of acid to a titanium oxide membrane which forms apatite having same Ca/P atomic ratio to a bone of mammal from the body liquid of mammal.

2. The titanium oxide-organic polymer hybrid material for an artificial bone of claim 1, wherein the organic polymer contains hydroxyl group and/or derivatives thereof, thiol group, aldehyde group or amino group.

3. The titanium oxide-organic polymer hybrid material for an artificial bone of claim 2, wherein the organic polymer composing the substrate is ethylene-polyvinyl alcohol copolymer.

4. The titanium oxide-organic polymer hybrid material for an artificial bone of claim 1, wherein the substrate composed by the organic polymer is treated with a denaturing agent composed of a silane coupling agent which forms Si—OH group on the surface of said substrate.

5. The titanium oxide-organic polymer hybrid material for an artificial bone of claim 4, wherein the organic polymer composing the substrate is the organic polymer containing hydroxyl group and/or derivatives thereof, thiol group, aldehyde group or amino group.

6. The titanium oxide-organic polymer hybrid material for an artificial bone of claim 5, wherein the substrate composed by the organic polymer is composed of ethylene-polyvinyl alcohol copolymer.

7. The titanium oxide-organic polymer hybrid material for an artificial bone of claim 4, wherein the silane coupling agent which forms Si—OH group on the surface of substrate is the compound represented by general formula 1,
R¹Si(—O—R²)(—O—R³)(—O—R⁴) general formula 1
wherein, R¹ is isocyanate group, epoxy group, vinyl group or hydro carbon group possessing chloride group, R², R³ or R⁴ are methoxy group or ethoxy group.

8. The titanium oxide-organic polymer hybrid material for an artificial bone of claim 7, wherein the organic polymer composing the substrate is the organic polymer containing hydroxyl group and/or derivatives thereof, thiol group, aldehyde group or amino group.

9. The titanium oxide-organic polymer hybrid material for an artificial bone of claim 8, wherein the substrate composed by the organic polymer is composed of ethylene-polyvinyl alcohol copolymer.

10. The titanium oxide-organic polymer hybrid material for an artificial bone of claim 1, wherein the treating of said titania gel with hot water or aqueous solution of acid is carried out by the acid concentration of pH7 or less that forms titania membrane possessing Ti—OH group in anatase fine crystal and/or 1 hour to 1 month period and/or 30° C. to 120° C. temperature.

11. The titanium oxide-organic polymer hybrid material for an artificial bone of claim 2, wherein the treating of said titania gel with hot water or aqueous solution of acid is carried out by the acid concentration of pH7 or less that forms titania membrane possessing Ti—OH group in anatase fine crystal and/or 1 hour to 1 month period and/or 30° C. to 120° C. temperature.

12. The titanium oxide-organic polymer hybrid material for an artificial bone of claim 1, wherein the apatite layer is foamed on the surface by contacting with supersaturated aqueous solution with respect to the apatite.

13. The titanium oxide-organic polymer hybrid material for an artificial bone of claim 2, wherein the apatite layer is formed on the surface by contacting with supersaturated aqueous solution with respect to the apatite.

14. The titanium oxide-organic polymer hybrid material for an artificial bone of claim 13, wherein the organic polymer composing the substrate is ethylene-polyvinyl alcohol copolymer.

15. The titanium oxide-organic polymer hybrid material for an artificial bone of claim 12, wherein the substrate composed by the organic polymer is treated with a denaturing agent composed of a silane coupling agent which forms Si—OH group on the surface of said substrate.

16. The titanium oxide-organic polymer hybrid material for an artificial bone of claim 15, wherein substrate composed by the organic polymer is composed by the organic polymer containing hydroxyl group and/or derivatives thereof, thiol group, aldehyde group or amino group.

17. The titanium oxide-organic polymer hybrid material for an artificial bone of claim 16, wherein the substrate composed by the organic polymer is composed of ethylene-polyvinyl alcohol copolymer.

18. The titanium oxide-organic polymer hybrid material for an artificial bone of claim 15, wherein the silane coupling agent which forms Si—OH group on the surface of substrate is the compound represented by general formula 1,
R¹Si(—O—R²)(—O—R³)(—O—R⁴) general formula 1
wherein, R¹ is isocyanate group, epoxy group, vinyl group or hydro carbon group possessing chloride group, R², R³ or R⁴ are methoxy group or ethoxy group.

19. The titanium oxide-organic polymer hybrid material for an artificial bone of claim 18, wherein the organic polymer composing the substrate is the organic polymer containing hydroxyl group and/or derivatives thereof, thiol group, aldehyde group or amino group.

20. The titanium oxide-organic polymer hybrid material for an artificial bone of claim 19, wherein the substrate composed by the organic polymer is composed of ethylene-polyvinyl alcohol copolymer.

21. The titanium oxide-organic polymer hybrid material for an artificial bone of claim 12, wherein the surface on which an apatite layer is formed by contacting with supersaturated aqueous solution with respect to the apatite is prepared by treatment of the titania gel with hot water or aqueous solution of acid, wherein said treatment is carried out by the acid concentration of pH7 or less that forms titania membrane possessing Ti—OH group in anatase fine crystal and/or 1 hour to 1 month period and/or 30° C. to 120° C. temperature.

22. The titanium oxide-organic polymer hybrid material for an artificial bone of claim 21, wherein the organic polymer composing the substrate is the organic polymer containing hydroxyl group and/or derivatives thereof, thiol group, aldehyde group or amino group.

23. The titanium oxide-organic polymer hybrid material for an artificial bone of claim 22, wherein the organic polymer composing the substrate is ethylene-polyvinyl alcohol copolymer.

24. The titanium oxide-organic polymer hybrid material for an artificial bone of claim 21, wherein the substrate composed by the organic polymer is treated with a denaturing agent composed of a silane coupling agent which forms Si—OH group on the surface of said substrate.

25. The titanium oxide-organic polymer hybrid material for an artificial bone of claim 24, wherein substrate composed by the organic polymer is composed by the organic polymer containing hydroxyl group and/or derivatives thereof, thiol group, aldehyde group or amino group.

26. The titanium oxide-organic polymer hybrid material for an artificial bone of claim 25, wherein the substrate composed by the organic polymer is composed of ethylene-polyvinyl alcohol copolymer.

27. The titanium oxide-organic polymer hybrid material for an artificial bone of claim 24, wherein the silane coupling agent which forms Si—OH group on the surface of substrate is the compound represented by general formula 1,
R¹Si(—O—R²)(—O—R³)(—O—R⁴) general formula 1
wherein, R¹ is isocyanate group, epoxy group, vinyl group or hydro carbon group possessing chloride group, R², R³ or R⁴ are methoxy group or ethoxy group.

28. The titanium oxide-organic polymer hybrid material for an artificial bone of claim 27, wherein substrate composed by the organic polymer is composed by the organic polymer containing hydroxyl group and/or derivatives thereof, thiol group, aldehyde group or amino group.

29. The titanium oxide-organic polymer hybrid material for an artificial bone of claim 28, wherein the substrate composed by the organic polymer is composed of ethylene-polyvinyl alcohol copolymer.

30. A bioactive organic/inorganic hybrid material obtained by bonding an organic polymer obtained by treating polymer-titanium oxide hybrid having polysiloxane obtained via sol prepared by hydrolysis/polycondensation of titanium alkoxide or polyalkyleneoxide chain possessing alkoxysilyl end and alkylene group represented by formula —(CH₂)n-, wherein n is an integer of 1 or bigger, so as to generate anatase type titanium oxide fine crystal with anatase type fine crystalline titanium dioxide by molecular level, under the presence of organic polysiloxane having silanol end and/or polymer having polyalkyleneoxide chain possessing alkoxysilyl end and alkylene group represented by formula —(CH₂)n-, wherein n is an integer of 1 or bigger, or adding solvent in case of need.

31. The bioactive organic/inorganic hybrid material obtained by bonding said organic polymer with anatase type fine crystalline titanium dioxide by molecular level of claim 30, wherein the treatment to generate anatase type titanium oxide fine crystal is to dip the substrate into hot water of 30° C. to 120° C. temperature or aqueous solution of acid.

32. The bioactive organic/inorganic hybrid material prepared by forming an apatite layer on the surface of the bioactive organic/inorganic hybrid material of claim 30 by contacting with supersaturated aqueous solution with respect to the apatite.

33. The bioactive organic/inorganic hybrid material prepared by forming an apatite layer on the surface of the bioactive organic/inorganic hybrid material of claim 31 by contacting with supersaturated aqueous solution with respect to the apatite.

34. Use of bioactive organic/inorganic hybrid material of claim 30 as a bone substitution material.

35. Use of bioactive organic/inorganic hybrid material of claim 31 as a bone substitution material.

36. Use of bioactive organic/inorganic hybrid material of claim 32 as a bone substitution material.

37. Use of bioactive organic/inorganic hybrid material of claim 33 as a bone substitution material.

38. Use of the bioactive organic/inorganic hybrid material obtained via sol prepared by hydrolysis/polycondensation of titanium alkoxide under the presence of organic polysiloxane having silanol end and/or polymer having alkoxysilyl end and polyalkyleneoxide chain as a bone repairing material.

39. A method for preparation of the bioactive organic/inorganic hybrid material comprising, bonding an organic polymer obtained by treating polymer-titanium oxide hybrid having polysiloxane obtained via sol prepared by hydrolysis/polycondensation of titanium alkoxide or polyalkyleneoxide chain possessing alkoxysilyl end and alkylene group represented by formula —(CH₂)n-, wherein n is an integer of 1 or bigger, so as to generate anatase type titanium oxide fine crystal with anatase type fine crystalline titanium dioxide by molecular level, under the presence of organic polysiloxane having silanol end and/or polymer having polyalkyleneoxide chain possessing alkoxysilyl end and alkylene group represented by formula —(CH₂)n-, wherein n is an integer of 1 or bigger, or adding solvent in case of need.

40. The method for preparation of the bioactive organic/inorganic hybrid material characterized by bonding the organic polymer of claim 30 with anatase type fine crystalline titanium dioxide by molecular level, wherein the treatment to generate anatase type titanium oxide fine crystal is to dip the substrate into hot water or aqueous solution of acid.

41. A method for preparation of the bioactive organic/inorganic hybrid material comprising, preparing sol or sol solution by hydrolysis/polycondensation of titanium alkoxide under the presence of organic polysiloxane having silanol end and/or polymer having polyalkyleneoxide chain possessing alkoxysilyl end and alkylene group represented by formula —(CH₂)n-, wherein n is an integer of 1 or bigger, or adding solvent in case of need, generating polymer-titanium dioxide hybrid having polyalkyleneoxide chain possessing alkoxysilyl end and alkylene group represented by formula —(CH₂)n-, wherein n is an integer of 1 or bigger, then preparing the bioactive organic/inorganic hybrid material characterized by bonding organic polymer with anatase type fine crystalline titanium dioxide by molecular level by the treatment to generate anatase type titanium oxide fine crystal and forming apatite on the surface of said bioactive organic/inorganic hybrid material by dipping into supersatirated aqueous solution with respect to the apatite.
